SEQUENCE LISTING (29 sequences)

SEQ ID NO: 1 - RNA, 20 nt, Homo sapiens
  source 1..20: mol_type = other RNA
tggctgcgtaccacacccgt

SEQ ID NO: 2 - RNA, 20 nt, Homo sapiens
  source 1..20: mol_type = other RNA
aagtggctgcgtaccacacc

SEQ ID NO: 3 - RNA, 20 nt, Homo sapiens
  source 1..20: mol_type = other RNA
gtgggtactcgaagtggctg

SEQ ID NO: 4 - RNA, 21 nt, Homo sapiens
  source 1..21: mol_type = other RNA
actgtgggtactcgaagtggc

SEQ ID NO: 5 - RNA, 20 nt, Homo sapiens
  source 1..20: mol_type = other RNA
ggtgtggtacgcagccactt

SEQ ID NO: 6 - RNA, 21 nt, Homo sapiens
  source 1..21: mol_type = other RNA
gtgtggtacgcagccacttcg

SEQ ID NO: 7 - RNA, 20 nt, Homo sapiens
  source 1..20: mol_type = other RNA
cagccacttcgagtacccac

SEQ ID NO: 8 - RNA, 20 nt, Homo sapiens
  source 1..20: mol_type = other RNA
gtactgtgggtactcgaagt

SEQ ID NO: 9 - RNA, 23 nt, Homo sapiens
  source 1..23: mol_type = other RNA
gacgggtgtggtacgcagccact

SEQ ID NO: 10 - DNA, 97 bp, synthetic construct
  source 1..97: mol_type = other DNA
gtgtggtacgcagccacttcggttttagagctagaaatagcaagttaaaataaggctagtccgttatcaacttgaaaaagtggcaccgagtcggtgc

SEQ ID NO: 11 - DNA, 105 bp, synthetic construct
  source 1..105: mol_type = other DNA
gacgggtgtggtacgcagccactgtttcagtactctggaaacagaatctactgaaacaaggcaaaatgccgtgtttatctcgtcaacttgttggcgagatttttt

SEQ ID NO: 12 - DNA, 24 bp, Homo sapiens
  source 1..24: mol_type = genomic DNA
ggtcagaacccagagtcatccagc

SEQ ID NO: 13 - DNA, 25 bp, Homo sapiens
  source 1..25: mol_type = genomic DNA
agaggtgtagagggtgctggtgaag

SEQ ID NO: 14 - RNA, 20 nt, Homo sapiens
  source 1..20: mol_type = other RNA
aggaagcactatggcatcta

SEQ ID NO: 15 - RNA, 20 nt, Homo sapiens
  source 1..20: mol_type = other RNA
gcagcagcaggaagcactat

SEQ ID NO: 16 - RNA, 20 nt, Homo sapiens
  source 1..20: mol_type = other RNA
agcagcagcaggaagcacta

SEQ ID NO: 17 - RNA, 20 nt, Homo sapiens
  source 1..20: mol_type = other RNA
cggagacagcagcagcagga

SEQ ID NO: 18 - RNA, 20 nt, Homo sapiens
  source 1..20: mol_type = other RNA
gtgcggagacagcagcagca

SEQ ID NO: 19 - RNA, 20 nt, Homo sapiens
  source 1..20: mol_type = other RNA
gccatagtgcttcctgctgc

SEQ ID NO: 20 - RNA, 20 nt, Homo sapiens
  source 1..20: mol_type = other RNA
atagtgcttcctgctgctgc

SEQ ID NO: 21 - RNA, 20 nt, Homo sapiens
  source 1..20: mol_type = other RNA
cttcctgctgctgctgtctc

SEQ ID NO: 22 - DNA, 23 bp, Homo sapiens
  source 1..23: mol_type = genomic DNA
atgcacagtgagggagacacttc

SEQ ID NO: 23 - DNA, 20 bp, Homo sapiens
  source 1..20: mol_type = genomic DNA
caccgcactcacctgtagat

SEQ ID NO: 24 - DNA, 82 bp, synthetic construct
  source 1..82: mol_type = other DNA
gtttcagtactctggaaacagaatctactgaaacaaggcaaaatgccgtgtttatctcgtcaacttgttggcgagatttttt

SEQ ID NO: 25 - DNA, 44 bp, Homo sapiens
  source 1..44: mol_type = genomic DNA
gcgacgggtgtggtacgcagccacttcgagtacccacagtacta

SEQ ID NO: 26 - DNA, 44 bp, Homo sapiens
  source 1..44: mol_type = genomic DNA
tagtactgtgggtactcgaagtggctgcgtaccacacccgtcgc

SEQ ID NO: 27 - DNA, 44 bp, Homo sapiens
  source 1..44: mol_type = genomic DNA
cgtgtgcggagacagcagcagcaggaagcactatggcatctatg

SEQ ID NO: 28 - DNA, 44 bp, Homo sapiens
  source 1..44: mol_type = genomic DNA
catagatgccatagtgcttcctgctgctgctgtctccgcacacg

SEQ ID NO: 29 - DNA, 76 bp, synthetic construct
  source 1..76: mol_type = other DNA
gttttagagctagaaatagcaagttaaaataaggctagtccgttatcaacttgaaaaagtggcaccgagtcggtgc

(19) United States
(12) Patent Application Publication
(10) Pub. No.: US 2026/0048074 A1
(43) Pub. Date: Feb. 19, 2026

(54) AGENT FOR TREATING OR PREVENTING A DOMINANTLY-INHERITED DISEASE

(71) Applicant: THE UNIVERSITY OF ADELAIDE, Adelaide, South Australia (AU)

(72) Inventors: Paul Quinton THOMAS, Mitcham, South Australia (AU); Fatwa ADIKUSUMA, Ferryden Park, South Australia (AU); Ashleigh Bryar GEIGER, Clearview, South Australia (AU)

(21) Appl. No.: 19/103,697

(22) PCT Filed: Aug. 15, 2023
(86) PCT No.: PCT/AU2023/050768

§ 371 Date: Feb. 13, 2025

(30) Foreign Application Priority Data
Aug. 16, 2022 (AU) 2022902322

(51) Int. Cl. A61K 31/7088; A61K 48/00; A61P 27/02; C12N 9/22; C12N 15/113; C12N 15/86

(52) U.S. Cl. CPC A61K 31/7088; A61K 48/005; A61P 27/02; C12N 9/226; C12N 15/1138; C12N 15/86; C12N 2310/20; C12N 2320/34

(57) ABSTRACT

An agent for treating a common form of autosomal dominant retinitis pigmentosa (ADRP) is disclosed, wherein the agent comprises a first nucleotide sequence encoding a CRISPR-associated (Cas) endonuclease which binds to an NG or NNGRRT PAM (protospacer adjacent motif) sequence, and a second nucleotide sequence encoding or comprising a guide RNA (gRNA) capable of forming a CRISPR-Cas complex with said Cas endonuclease, wherein the gRNA is specifically targeted to a target mutant allele selected from RHOP23H and NR2E3G56R.

### TECHNICAL FIELD

The present disclosure relates particularly to an agent for treating certain forms of autosomal dominant retinitis pigmentosa (ADRP).

### PRIORITY DOCUMENT

The present application claims priority from Australian Provisional Patent Application No. 2022902322 titled “AGENT FOR TREATING OR PREVENTING A DOMINANTLY-INHERITED DISEASE” and filed on 16 Aug. 2022, the content of which is hereby incorporated by reference in its entirety.

### BACKGROUND

Retinitis pigmentosa (RP) is a leading cause of inherited blindness characterised by rod photoreceptor cell death that leads to a reduced ability of the eye to adapt to dim light or to the dark (“night blindness”), peripheral vision loss and, eventually (over a period of decades), to loss of central vision. It has been estimated that between about 1.77 and 2.35 million people are affected worldwide (https://rarediseases.org/rare-diseases/retinitis-pigmentosa/), and about 25% to 30% of cases are inherited in an autosomal dominant fashion.

The two most common sites for mutations causing autosomal dominant retinitis pigmentosa (ADRP) are the genes for rhodopsin (RHO) and nuclear receptor subfamily two group E member 3(NR2E3). RHO is the most abundant protein in retinal photoreceptor cells (comprising almost 50% of the total protein in the rod outer segments) and is involved in the photo-transduction cascade in rod photoreceptor cells, while NR2E3 is a photoreceptor-specific transcription factor that is key to the development and maintenance of rod photoreceptors. In complex with other factors, NR2E3 promotes the transcription of rod genes including RHO. There are over 150 known mutations in RHO which lead to ADRP (www.ncbi.nlm.nih.gov/clinvar), but only one ADRP-causative mutation in the NR2E3 gene, namely a c.166G>A (p.Gly56Arg; G56R) mutation in NR2E3. This, however, is the second most common mutation for ADRP. The most common ADRP-causing mutation is a c.68C>A (p.Pro23His; P23H) mutation in RHO.

Unfortunately, ADRP is currently incurable and nor is there any available treatments to prevent or significantly retard the progression of the disease, with perhaps only Vitamin A palmitate supplementation providing any evidence of being able to slow the decline of photoreceptor function (in children) (Berson, E L et al., JAMA Ophthalmol 136(5):490-495, 2018). Other than that, the only other options at present are essentially limited to the use, in the later stages of the disease, of acetazolamide to reduce swelling in the retina (macular oedema) caused by the disease to improve vision (Fishman G A et al., Arch Ophthalmol 107(10):1445-1452, 1989), or to undergo a retinal implant (again during the later stages of the disease) to provide some partial sight. Accordingly, new and innovative therapeutic approaches are desperately needed to treat this devastating disease.

One potential approach to the development of new ADRP therapies involves the use of genome editing technology, including Clustered Regularly Interspaced Short Palindromic Repeats (CRISPR) methodologies which are recognised as offering enormous potential for the treatment of genetic diseases. Indeed, clinical trials using CRISPR methodologies have recently commenced for a range of different diseases including sickle cell disease and hereditary transthyretin amyloidosis (hATTR) (https://innovativegenomics.org/news/crispr-clinical-trials-2021/). In addition, and of particular interest in the context of the present disclosure, in 2020, a clinical trial of a CRISPR-based therapy commenced with patients suffering from a recessive retinopathy, namely Leber congenital amaurosis (LCA), which causes severe vision loss or blindness in newborns (ClinicalTrials.gov Identifier: NCT03872479; and Maeder M et al., Nat Med 25:229-233, 2019).

However, a major limitation of the use of CRISPR methodologies in the context of a possible therapy for dominantly-inherited genetic diseases such as ADRP is the need to specifically target the disease-causing allele only, which is particularly challenging for single nucleotide changes (i.e. point mutations) in coding sequences. In addition, in cases where gene editing is contemplated of a dominantly-inherited genetic disease, it may generally be preferred to aim to correct the mutant allele by exploiting homology directed repair (HDR). However, since the HDR process takes place during the S/G2 phase of dividing cells (Jasin M et al., Cold Spring Harb Perspect Biol 5:a012740, 2013), this can be difficult in post-mitotic cells such as retinal photoreceptor cells (Cai Y et al., Sci Adv 5:eaav3335, 2019).

Thus, in the work leading to the present disclosure, the Applicant looked to determine whether a CRISPR-based methodology could be developed that could provide a potentially viable therapy for ADRP without needing to rely on a repair process such as HDR.

### SUMMARY

According to a first aspect, the present disclosure relates to an agent for treating a common form of autosomal dominant retinitis pigmentosa (ADRP) in a subject, wherein said agent comprises:
(i) a first nucleotide sequence encoding a CRISPR-associated (Cas) endonuclease which binds to an NG or NNGRRT PAM (protospacer adjacent motif) sequence; and
(ii) a second nucleotide sequence encoding or comprising a guide RNA (gRNA) capable of forming a CRISPR-Cas complex with said Cas endonuclease, wherein said gRNA is specifically targeted to a target mutant allele selected from RHOP23H and NR2E3G56R.

Upon delivery of the agent to a suitable cell (particularly, a retinal photoreceptor cell) of a subject afflicted with, or predisposed to, ADRP caused by the RHOP23H or NR2E3G56R allele, a CRISPR-Cas complex (a ribonucleoprotein complex; RNP) is formed from expressed Cas endonuclease and the gRNA which is capable of specifically targeting and cleaving the target mutant allele, to result in the inactivation of the target mutant allele (i.e. such that the encoded mutant protein can not be expressed).

In some preferred embodiments, the first nucleotide sequence may encode any Cas endonuclease which binds to the PAM sequence NG. One suitable Cas endonuclease is known as SpCas9-NG.

In some preferred embodiments, the second nucleotide sequence may encode or comprise a gRNA which comprises a targeting nucleotide sequence selected from SEQ ID NOs:3, 6, 8 and 9 (where the target mutant allele is RHOP23H) or SEQ ID NOs:17 and 18 (where the target mutant allele is NR2E3G56R).

In a second aspect, the present disclosure provides a pharmaceutical composition comprising an agent according to the first aspect in combination with a pharmaceutically acceptable carrier, diluent and/or excipient.

In a third aspect, the present disclosure provides a method of treating a subject afflicted with, or predisposed to, ADRP, wherein the method comprises administering the agent of the first aspect or a pharmaceutical composition of the second aspect to the said subject.

In some embodiments, the method is commenced during childhood such as, for example, before ADRP disease onset and/or the appearance of ADRP symptoms, the child subject having been genotyped for the presence of the RHOP23H and NR2E3G56R mutant allele. Such early commencement of the method may prevent or delay ADRP disease onset or the appearance of ADRP symptoms.

In a fourth aspect, the present disclosure provides the use of the agent of the first aspect for treating or preventing ADRP caused by a RHOP23H or NR2E3G56R mutant allele.

In a fifth aspect, the present disclosure provides the use of the agent of the first aspect in the manufacture of a medicament (e.g. a pharmaceutical composition) for treating or preventing ADRP caused by a RHOP23H or NR2E3G56R mutant allele.

In a sixth aspect, the present disclosure provides a CRISPR-Cas complex (a ribonucleoprotein complex; RNP) comprising a CRISPR-associated (Cas) endonuclease which binds to an NG or NNGRRT PAM (protospacer adjacent motif) sequence and a guide RNA (gRNA), wherein said gRNA is specifically targeted to a target mutant allele selected from RHOP23H and NR2E3G56R.

### BRIEF DESCRIPTION OF FIGURES

FIG. 1 provides: (A) a schematic representation of ten (10) guide RNA molecules (gRNAs) designed to specifically target the RHOP23H mutant allele causative of ADRP. The P23H mutation (i.e. c.68C>A) is shown in the allelic DNA sequence with a black background. The relevant PAM sequences are located immediately adjacent to the depicted point of the respective gRNA molecules; for instance, the NG PAM sequence for the gRNA designated “RhoNG1” comprises the “CG” motif immediately downstream of the final nucleotide (“T”) of the allelic DNA sequence targeted by this gRNA. RhoNG5 and rhoVQR share the same gRNA sequence and target the overlapping PAM motifs of “CG” and “CGA” respectively; and (B) graphical results of CRISPR activities on the RHOP23H and wild type (WT) alleles in HEK293T^{RHOP23H/+} disease model cells using four (4) gRNAs, namely rhoNG3, rhoNG6, rhoNG8 and rhoSA1. The graph shows the percentage of WT, P23H and indel alleles (i.e. RHOP23H alleles that have been edited) after transfection and selection of HEK293T^{RHOP23H/+} (average of n=3 biological replicates). It is considered that the WT proportion of >33% is likely due to large deletions which do not amplify due to loss of primer site(s) (Adikusuma F et al., Nature 560(7717):E8, 2018); and

FIG. 2 provides: (A) a schematic representation of nine (9) guide RNA molecules (gRNAs) designed to specifically target the NR2E3G56R allele causative of ADRP. The G56R mutation (i.e. c.166G>A) is shown in the allelic DNA sequence with a black background. The relevant PAM sequences are located immediately adjacent to the point of the respective gRNA molecules; for instance, the NG PAM sequence for the gRNA designated “nrNG1” comprises the “TG” motif immediately downstream of the final nucleotide (“A”) of the allelic DNA sequence targeted by this gRNA. The gRNAs nrNG3 and SpCas9-3 share the same gRNA sequence and target the PAM motifs of “TG” and “TGG” respectively; and (B) graphical results of CRISPR activities on the NR2E3G56R and wild type (WT) alleles in HEK293T^{NR2E3G56R/+} disease model cells using two (2) gRNAs, namely nrNG4 and nrNG5. The graph shows the percentage of WT, G56R and indel alleles (i.e. NR2E3G56R alleles that have been edited) after transfection and selection of HEK293T^{NR2E3G56R/+} (average of n=3 biological replicates). The >50% WT proportion is likely due to large deletions which do not amplify due to loss of primer site(s) (Adikusuma F et al., Nature 560(7717):E8, 2018).

### DETAILED DESCRIPTION

The two most common mutations causing autosomal dominant retinitis pigmentosa (ADRP) are both “gain-of-function” mutations in a single allele (Diakatou M et al., Int J Mol Sci 20:2542, 2019). In particular, the RHO disease-variant P23H causes progressive loss of vision via a toxic gain-of-function mechanism (Athanasiou D et al., Prog Retin Eye Res 62:1-23, 2018), wherein the mutation causes misfolded RHO-P23H protein to accumulate in rod photoreceptor cells, overwhelming the proteasome and resulting in the gradual toxic accumulation of RHO-P23H, which causes retinal degeneration. Similarly, the NR2E3 disease-variant G56R results in the expression of a mutant protein which functions as a gain-of-function protein, and which appears to interact with the CRX cofactor protein to prevent expression of rod-specific genes (Roduit R et al., PLoS One 4:e7379, 2009) essential for rod cell differentiation. Recognising that both of the affected genes, RHO and NR2E3, are haplosufficient (i.e. one normal copy of the gene is sufficient for normal vision (Diakatou et al., 2019 supra; and Haider N B et al., Nat Genet 24:127-131, 2000)), the Applicant looked to determine whether a CRISPR-based methodology could be developed that could specifically ablate (inactivate) the causative mutant allele so as to prevent expression of the mutant proteins and thereby provide a potentially viable therapy for ADRP (which may halt or even reverse the retinal degeneration in affected patients). Desirably, the normal copy of the respective gene provides for “disease rescue” (i.e. phenotypic rescue via haplosufficiency) such that the strategy may be effective without needing to rely on a repair process such as HDR.

Thus, in a first aspect, the present invention provides an agent for treating a common form of autosomal dominant retinitis pigmentosa (ADRP) in a subject, wherein said agent comprises:
(i) a first nucleotide sequence encoding a CRISPR-associated (Cas) endonuclease which binds to an NG or NNGRRT PAM (protospacer adjacent motif) sequence; and
(ii) a second nucleotide sequence encoding or comprising a guide RNA (gRNA) capable of forming a CRISPR-Cas complex with said Cas endonuclease, wherein said gRNA is specifically targeted to a target mutant allele selected from RHOP23H and NR2E3G56R.

In some embodiments, the agent may comprise one or two polynucleotide molecules such as expression vectors/constructs and/or viral vectors suitable for delivery to the subject (e.g. particularly to a photoreceptor cell of the subject), such that the first and second nucleotide sequences (and operably linked promoter and/or regulatory sequences) may be provided on the same or different polynucleotide molecules. In such embodiments, the agent therefore comprises: (i) a first nucleotide sequence encoding a Cas endonuclease which binds to an NG or NNGRRT protospacer adjacent motif sequence, wherein said first nucleotide sequence is operably linked to a first promoter and/or regulatory sequence(s) for expression of said Cas endonuclease; and (ii) a second nucleotide sequence encoding a guide RNA (gRNA) capable of forming a CRISPR-Cas complex with said Cas endonuclease, wherein said second nucleotide sequence is operably linked to a second promoter and/or regulatory sequence(s) for transcription of said gRNA, and wherein said gRNA is specifically targeted to a target mutant allele selected from RHOP23H and NR2E3G56R. The first and second promoter and/or regulatory sequences may be the same or different. Upon delivery of such an agent to a suitable target cell of a subject afflicted with, or predisposed to (i.e. the subject has the RHOP23H or NR2E3G56R allele), ADRP. particularly a retinal photoreceptor cell, the Cas endonuclease is expressed within the cell along with the production of the gRNA by transcription, to enable formation of a CRISPR-Cas complex capable of specifically targeting and cleaving (cutting) in both DNA strands of the target mutant allele, to result in the inactivation of the target mutant allele (i.e. such that the encoded mutant protein can not be expressed).

However, it is not necessary for both the Cas endonuclease to be expressed and the gRNA to be transcribed in the target cell. That is, the agent could comprise a first nucleotide sequence which is comprised of mRNA for expression of the Cas endonuclease within the cell, or the agent could comprise a second nucleotide sequence which comprises the gRNA per se. Thus, in some other embodiments, the agent comprises: (i) a first nucleotide sequence encoding a CRISPR-associated (Cas) endonuclease which binds to an NG or NNGRRT protospacer adjacent motif sequence (e.g. the first nucleotide sequence is provided as mRNA for expression of the Cas endonuclease); and (ii) a second nucleotide sequence encoding a guide RNA (gRNA) capable of forming a CRISPR-Cas complex with said Cas endonuclease, said second nucleotide sequence operably linked to a promoter and/or regulatory sequence(s) for transcription of said gRNA, and wherein said gRNA is specifically targeted to a target mutant allele selected from RHOP23H and NR2E3G56R. And in some yet other embodiments, the agent comprises: (i) a first nucleotide sequence encoding a CRISPR-associated (Cas) endonuclease which binds to an NG or NNGRRT PAM (protospacer adjacent motif) sequence, said first nucleotide sequence operably linked to a promoter and/or regulatory sequence(s) for expression of said Cas endonuclease; and (ii) a second nucleotide sequence comprising a guide RNA (gRNA) capable of forming a CRISPR-Cas complex with said Cas endonuclease, wherein said gRNA is specifically targeted to a target mutant allele selected from RHOP23H and NR2E3G56R.

However the cell is provided with the CRISPR-Cas complex, and while not wishing to be bound by theory, it is considered that the CRISPR-Cas complex facilitates the incorporation of frame-shifting indels (i.e. the “insertion or deletion” of nucleotides) via non-homologous or microhomology-mediated end-joining repair mechanisms (NHEJ/MMEJ) after a double-stranded DNA break (cleavage), and in this way, it is considered that the target mutant allele (i.e. the RHOP23H or NR2E3G56R) is inactivated without needing to rely on a repair process such as HDR, permitting phenotypic rescue via haplosufficiency.

In some preferred embodiments, the first nucleotide sequence may encode any Cas endonuclease which binds to the PAM sequence NG. One suitable Cas endonuclease of this type is known as SpCas9-NG described in, for example, Nishimasu H et al., Science 361:1259-1262, 2018.

In some other preferred embodiments, the first nucleotide sequence may encode any Cas endonuclease which binds to the PAM sequence NNGRRT. One suitable Cas endonuclease of this type is known as SaCas9 described in, for example, Ran F A et al., Nature 520(7546):186-191, 2015, and Maeder et al., 2019 supra.

The second nucleotide sequence may encode or comprise a gRNA comprising a targeting nucleotide sequence (otherwise known as the gRNA spacer sequence, which is typically of ˜20 nucleotides in length and defines the genomic sequence to be targeted).

In some embodiments, the second nucleotide sequence may encode or comprise a gRNA with a targeting nucleotide sequence which hybridises to a nucleotide sequence within the target mutant allele which includes the site of mutation, and wherein the site of mutation corresponds to at least a nucleotide located at or near (e.g. within 8 or less nucleotides, and more preferably, within 6 or less nucleotides), of the 3′-end of the gRNA. As shown hereinafter in the Example(s), such embodiments may be particularly suited to the targeting of RHOP23H.

In some embodiments, the second nucleotide sequence may encode or comprise a gRNA with a targeting nucleotide sequence selected from those shown hereinafter as:

| | |
|---|---|
| For targeting RHOP23H | SEQ ID NOs: 1-9 |
| For targeting NR2E3G56R | SEQ ID NOs: 14-21 |

Preferably, where the target mutant allele is RHOP23H, the second nucleotide sequence encodes or comprises a gRNA comprising a targeting nucleotide sequence selected from SEQ ID NOs:3, 6, 8 and 9, more preferably from SEQ ID NOs:3, 6 and 8.

Preferably, where the target mutant allele is NR2E3G56R, the second nucleotide sequence encodes or comprises a gRNA comprising a targeting nucleotide sequence selected from SEQ ID NOs:17 and 18.

As will be known to those skilled in the art, the gRNA preferably further comprises a scaffold sequence necessary for binding to the Cas endonuclease (and thereby formation of the CRISPR-Cas complex), also known as the trans-activating RNA (tracrRNA). A gRNA comprising both a targeting nucleotide sequence and a scaffold sequence is known as a single guide RNA (sgRNA) molecule. Suitable scaffold sequences for use with SpCas9-NG are well-known to those skilled in the art and have been described in Nishimasu et al., 2018 supra; the disclosure(s) of which are incorporated herein by reference. Suitable scaffold sequences for use with SaCas9 are also well-known to those skilled in the art. However, a preferred scaffold for use with SaCas9, is as described hereinafter and comprises the nucleotide sequence shown below:

(SEQ ID NO: 24)
GTTTCAGTACTCTGGAAACAGAATCTACTGAAACAAGGCAAAATGCCGT
GTTTATCTCGTCAACTTGTTGGCGAGATTTTTT.

Thus, by way of example, where the target mutant allele is RHOP23H, the first nucleotide sequence may encode the Cas endonuclease known as SpCas9-NG, and the second nucleotide sequence may encode or comprise a gRNA comprising a suitable scaffold (e.g. GTTTTAGAGCTA GAAATAGCAAGTTAAAATAAGGCTAGTCCGTTATCAACTTGAAAAAGTGGCACCGAGTCGGTGC (SEQ ID NO:29) and a targeting nucleotide sequence with a nucleotide sequence selected from SEQ ID NOs:3, 6 and 8. In another example, again where the target mutant allele is RHOP23H, the first nucleotide sequence may encode the Cas endonuclease known as SaCas9, and the second nucleotide sequence may encode or comprise a gRNA comprising a scaffold with the nucleotide sequence shown as SEQ ID NO:24 and a targeting nucleotide sequence with the nucleotide sequence shown as SEQ ID NO:9.

The agent may be prepared for delivery to the subject in the form of a viral delivery vector or virus-like particles (VLPs) including engineered virus-like particles (eVLPs). Examples of suitable viral delivery vectors include the “typical” adeno-associated virus (AAV) vector (as described in, for example, Naso M F et al., BioDrugs 31(4):317-334, 2017; and Xu C L et al., Viruses 11(1):28, 2019), as well as other vectors based upon full length adenovirus (AdV), lentivirus (LV) vectors (e.g. non-integrating lentiviral vectors), and baculovirus (BV) vectors (Aulicinio F et al., Nucleic Acids Res 50(13); 7783-7799, 2022) which may, for example, be used with magnetic nanoparticles for complement shielding. Examples of suitable VLPs include those based upon retroviral capsids (e.g. VLPs based upon the gag polyprotein of Friend murine leukaemia virus (FMLV) which have been shown to be capable of delivering encapsulated macromolecules to the eye; Banksota S et al., Cell 185:250-265, 2022). However, the agent may also be prepared for delivery to the subject in other forms well-known to those skilled in the art, such as non-viral vector systems including liposomes, lipid nanoparticles (LNPs), delivery forms incorporating a cell-penetrating peptide (CPP), nanoparticles composed of polymeric or other organic materials, and nanoparticles composed of gold (auNPs), silica and/or other inert inorganic materials. The use of nanoparticle delivery forms may, for example, offer advantages in terms of reduced immunogenicity, flexibility in design, and ease of large scale production for therapeutic use. Suitable delivery vectors such as these and the viral vectors mentioned above have been reviewed in, for example, Lino C A et al., Drug Deliv 25(1):1234-1257, 2018; and Behr M et al., Acta Pharm Sin B 11(8):2150-2171, 2021; the disclosure(s) of which are incorporated herein by reference.

In some preferred embodiments, the agent is prepared for delivery to the subject in the form of an AAV delivery vector(s). AAVs have been routinely used for the in vivo delivery of various polynucleotide molecules for therapeutic purposes, including for the delivery of CRISPR-Cas systems to the eye (see Hung S S et al., Invest Ophthalmol Vis Sci 57:3470-3476, 2016; and Li F et al., Front Cell Neurosci 14:570917, 2020). In one particular embodiment, the first nucleotide sequence is provided on a first AAV vector (“vector 1”) operably linked to a suitable promoter sequence (e.g. a strong constitutive promoter sequence such as the well-known cytomegalovirus (CMV) promoter sequence) to drive expression of the Cas endonuclease, and the second nucleotide sequence is provided on a second AAV vector (“vector 2”) operably linked to a suitable promoter sequence (e.g. a strong constitutive promoter sequence such as the well-known human U6 promoter sequence) for the production of the gRNA.

In some other preferred embodiments, the agent is prepared for delivery to the subject in the form of an LV delivery vector(s). Recent work has indicated that a delivery vector based upon a self-inactivating non-integrating lentivirus can be successfully used for delivery of CRISPR-Cas systems to the eye (Ling S et al., Nature Biomed Eng 5:144-156, 2021) with very high efficiency. In this example, the LV delivery vector co-delivered to the target cell, mRNA encoding for the Cas endonuclease and an expression cassette (incorporated into the lentiviral genome) for production of the gRNA (by transcription) by the target cell. Such vectors characteristically produce a “burst” of Cas endonuclease expression (due to the rapid degradation of the mRNA in the cell) which may prevent or reduce the potential for off-target mutations. Where it is desired to express the Cas endonuclease and gRNA from respective expression cassettes within the target cell, LV delivery vectors may be advantageous in that their larger packaging capacity (i.e. as compared to AAVs; ˜8.0 kb vs. ˜4.7 kb) enables the generation and delivery of a single LV vector comprising both the first nucleotide sequence (encoding the Cas endonuclease) and the second nucleotide sequence (encoding the gRNA).

In a second aspect, the present disclosure provides a pharmaceutical composition comprising an agent according to the first aspect in combination with a pharmaceutically acceptable carrier, diluent and/or excipient.

Examples of suitable carriers and diluents are well-known to those skilled in the art, and are described in, for example, Remington's Pharmaceutical Sciences, Mack Publishing Co., Easton, PA 1995. Examples of suitable excipients may be found in the Handbook of Pharmaceutical Excipients, 2^{nd} Edition, (1994), Edited by A Wade and PJ Weller. Examples of suitable carriers include lactose, starch, glucose, methyl cellulose, magnesium stearate, mannitol, sorbitol and the like. Examples of suitable diluents include ethanol, glycerol and water.

The pharmaceutical composition may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, dyes and even flavouring agents may be provided in the pharmaceutical composition. Examples of preservatives include sodium benzoate, sorbic acid and esters of p-hydroxybenzoic acid. Anti-oxidants and suspending agents may be also used.

The pharmaceutical composition will be administered to a subject afflicted with, or predisposed to, ADRP in a therapeutically effective amount, that is an amount sufficient to effect beneficial or desired clinical results. A therapeutically effective amount can be administered in one or more administrations. Typically, a therapeutically effective amount will be sufficient for treating the ADRP or otherwise to palliate, ameliorate, stabilise, reverse, slow or delay the progression of the disease. Further, and notwithstanding the above, it will be understood by those skilled in the art that the therapeutically effective amount may vary and depend upon a variety of factors including the activity of the CRISPR-Cas system, the metabolic stability and length of action of the particular CRISPR-Cas system, the age, body weight, sex and/or health of the subject, the route and time of administration, and the severity of the ADRP to be treated.

The pharmaceutical compositions may preferably be intended for use as a “single application” therapy, however single daily administration, multiple daily administration, controlled or sustained release, or at regular intervals (e.g. every month, every 2 months or every three months) or irregular intervals (e.g. the period of the interval may, however, vary from time to time across the duration of the subject's treatment) are also contemplated as needed to achieve the most effective results. The pharmaceutical composition may be administered by, for example, sub-retinal or intravitreal injection (with or without in vivo electroporation by application of suitable square pulses/waves from a standard porator apparatus such as those available from BTX®, Holliston, MA, United States of America).

In a third aspect, the present disclosure provides a method of treating a subject afflicted with, or predisposed to, ADRP, wherein the method comprises administering the agent of the first aspect or a pharmaceutical composition of the second aspect to the said subject.

Preferably, the method is commenced upon diagnosis, disease onset and/or the appearance of ADRP symptoms, typically during adolescence, and may be continued for the remainder of the subject's life. In some embodiments, the method is commenced during childhood such as, for example, before disease onset and/or the appearance of ADRP symptoms, the child subject having been genotyped for the presence of the RHOP23H and NR2E3G56R mutant allele. Such early commencement of the method may prevent or delay disease onset or the appearance of symptoms.

In a fourth aspect, the present disclosure provides the use of the agent of the first aspect for treating or preventing ADRP caused by a RHOP23H or NR2E3G56R mutant allele.

In a fifth aspect, the present disclosure provides the use of the agent of the first aspect in the manufacture of a medicament (e.g. a pharmaceutical composition) for treating or preventing ADRP caused by a RHOP23H or NR2E3G56R mutant allele.

In a sixth aspect, the present disclosure provides a CRISPR-Cas complex (a ribonucleoprotein complex; RNP) comprising a CRISPR-associated (Cas) endonuclease which binds to an NG or NNGRRT PAM (protospacer adjacent motif) sequence and a guide RNA (gRNA), wherein said gRNA is specifically targeted to a target mutant allele selected from RHOP23H and NR2E3G56R.

The CRISPR-Cas complex of the sixth aspect may be useful as a “directly delivered” agent for treating autosomal dominant retinitis pigmentosa (ADRP) in a subject, wherein the ADRP-causative mutation is RHOP23H or NR2E3G56R. As such, the present disclosure is also to be understood as extending to, for example, a pharmaceutical composition comprising the CRISPR-Cas complex of the sixth aspect (in combination with a pharmaceutically acceptable carrier, diluent and/or excipient) and a method of treating a subject afflicted with, or predisposed to, ADRP, wherein the method comprises administering the CRISPR-Cas complex of the sixth aspect or a pharmaceutical composition comprising same. The Cas endonuclease of the CRISPR-Cas complex of the sixth aspect may be, for example, SpCas9-NG, SpCas9-VQR or SaCas9. The gRNA of the CRISPR-Cas complex of the sixth aspect may comprise a targeting nucleotide sequence (gRNA spacer sequence) selected from those shown hereinafter as SEQ ID NOs:1-9 (for targeting RHOP23H) and SEQ ID NOs:14-21 (for targeting NR2E3G56R). The gRNA may be produced synthetically and include one or more chemical modification to increase the stability of the gRNA and/or CRISPR-Cas complex (e.g. the incorporation of one or more nucleotide with a 2′0-methyl (2′OMe) or 2′-O-methoxyethyl (MOE) ribose modification, one or more 5′-methylcytosine (MeC) nucleotide, and/or one or more phosphorothioate (PS) backbone linkage). The CRISPR-Cas complex of the sixth aspect may be directly delivered to a target cell (particularly, the nucleus of a target cell) using one or more of various techniques well-known to those skilled in the art (for example, the techniques reviewed in Zhang S et al., Theranostics 11(2):614-648, 2021; the disclosure(s) of which are incorporated herein by reference), and include, for example, physical approaches such as microinjection, electroporation, biolistic and microfluidic techniques, and/or the use of synthetic carriers such as lipid nanoparticles (LNPs) and cell-derived vesicles, polymers, nanogels, inorganic nanoparticles and DNA nanoclews. In some embodiments, the CRISPR-Cas complex of the sixth aspect may be delivered to a target cell in the form of VLPs, and especially VLPs (or eVLPs) based upon retroviral capsids (e.g. FMLV-based eVLPs which have been shown to be capable of delivering encapsulated macromolecules to the eye; Banksota et al., 2022 supra).

In this specification, a number of terms are used which are well-known to those skilled in the art. Nevertheless, for the purposes of clarity, a number of these terms are hereinafter defined.

As used herein, the term “treating” includes prophylaxis as well as the alleviation of established symptoms of a disease or condition. As such, the act of “treating” ADRP therefore includes: (1) preventing or delaying the appearance of clinical symptoms of the disease or condition developing in a subject afflicted with or predisposed to the disease or condition; (2) inhibiting the disease or condition (i.e. arresting, reducing or delaying the development of the disease or condition or a relapse thereof (in case of a maintenance treatment) or at least one clinical or subclinical symptom thereof; and (3) relieving or attenuating the disease or condition (i.e. causing regression of the disease or condition or at least one of its clinical or subclinical symptoms).

As used herein, the phrase “manufacture of a medicament” includes the use of the agent directly as the medicament or in any stage of the manufacture of a medicament.

The agent of the present disclosure is hereinafter further described with reference to the following non-limiting examples and accompanying figures.

### EXAMPLES

### Example 1 CRISPR-Based Editing of an ADRP-Causing RHO Allele

### Identification of Candidate Guide RNAs

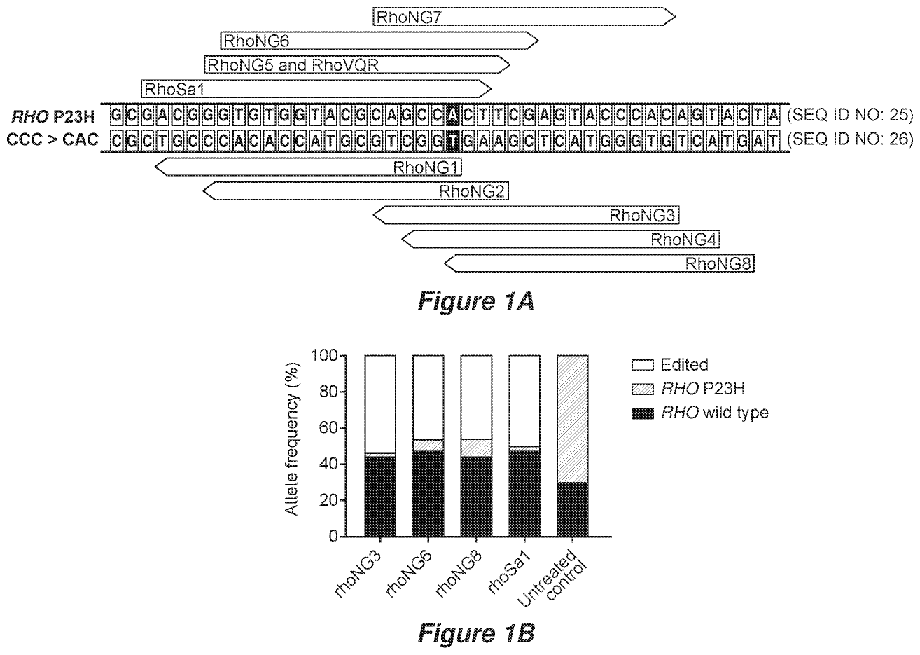

To identify candidate guide RNA (gRNA) sequences for allele-specific targeting, the human RHOP23H allele sequence was scanned for all possible CRISPR-Cas9 gRNA options. While it was found that there were no gRNA sequences targeting the P23H site available for the canonical SpCas9 system, gRNAs for three other Cas9 endonucleases were identified. In particular, eight (8) gRNAs for SpCas9-NG (also known simply as Cas9-NG) were identified (designated rhoNG1-8), one gRNA for SpCas9-VQR (designated rhoVQR1) and one gRNA for SaCas9 (designated rhoSA1). The short protospacer adjacent motif (PAM) sequence for SpCas9-NG is an NG PAM, while SpCas9-VQR binds to an NGA PAM, and SaCas9 binds to an NNGRRT PAM sequence. Where gRNAs of canonical length did not begin with G, an additional G was appended to the 5′ end of the gRNA to enhance transcription under the U6 RNA polymerase III promoter. In some instances, this G is naturally occurring in the target sequence (SEQ ID NOS:1, 2, 7, and 9); otherwise, the additional G is a mismatch with the target DNA sequence (SEQ ID NO:4). These RHO gRNAs are shown schematically in FIG. 1A and the allele-targeting sequences (i.e. gRNA spacer sequences) are as shown in Table 1.

TABLE 1

| gRNA | 5′→3′ targeting nucleotide sequence |
|---|---|
| rhoNG1* | UGGCUGCGUACCACACCCGU (SEQ ID NO: 1) |
| rhoNG2* | AAGUGGCUGCGUACCACACC (SEQ ID NO: 2) |
| rhoNG3 | GUGGGUACUCGAAGUGGCUG (SEQ ID NO: 3) |
| rhoNG4* | ACUGUGGGUACUCGAAGUGGC (SEQ ID NO: 4) |
| rhoNG5 | GGUGUGGUACGCAGCCACUU (SEQ ID NO: 5) |
| rhoNG6 | GUGUGGUACGCAGCCACUUCG (SEQ ID NO: 6) |
| rhoNG7* | CAGCCACUUCGAGUACCCAC (SEQ ID NO: 7) |
| rhoNG8 | GUACUGUGGGUACUCGAAGU (SEQ ID NO: 8) |
| rhoSA1 | GACGGGUGUGGUACGCAGCCACU (SEQ ID NO: 9) |
| rhoVQR | GGUGUGGUACGCAGCCACUU (SEQ ID NO: 5) |

(* An additional G nucleotide was added to the 5′ end of the given sequence to enable enhance transcription from the U6 promoter)

Screening of Candidate gRNAs for Off-Target Activity

Each of the ten candidate gRNA molecules were screened to ensure that their cleavage activity was limited to the RHOP23H allele sequence, and there was no substantial cleavage observed in the wild type (WT) allele.

### Construction of CRISPR Expression Constructs

### Cas9-NG Expression Constructs

Initially, a plasmid for expressing SpCas9-NG and including a puromycin resistance gene (PuroR) was constructed using standard methodologies known to those skilled in the art; hereafter Cas9-NG-puro. The nucleotide sequence for SpCas9-NG was as published (Nishimasu et al., 2018 supra). The inclusion of PuroR facilitates enrichment for transfected cells following in vitro transfection. Expression of the SpCas9-NG enzyme and PuroR is driven by the well-known CBh promoter (Gray SJ et al., Hum Gene Ther 9:1143-1153, 2011), such that puromycin resistance only occurs in tandem with SpCas9-NG expression. The plasmid also encodes a gRNA site upstream of the scaffold sequence, where the oligonucleotide sequence for a custom gRNA molecule can be inserted for expression of the gRNA by the U6 RNA polymerase III promoter. Subsequently, custom oligonucleotide sequences corresponding to the gRNAs in Table 1 (SEQ ID NOs:1-8) were inserted into the gRNA site of Cas9-NG-puro to generate eight RHOP23H-targeting expression constructs, according to standard methodologies known to those skilled in the art. An example of the nucleotide sequence encoding for an example of a full-length gRNA (comprising the RhoNG6 spacer sequence and the scaffold sequence) in a Cas9-NG-puro expression plasmid is shown below:

(SEQ ID NO: 10)
5′-GTGTGGTACGCAGCCACTTCGgttttagagctagaaatagcaagtt
aaaataaggctagtccgttatcaacttgaaaaagtggcaccgagtcggt
gc-3′

(Note. The RhoNG6 spacer sequence is shown in uppercase, while the scaffold sequence is shown lowercase.)

### Cas9-VQR Expression Construct

A plasmid for expressing SpCas9-VQR and PuroR was obtained (px459-VQR, Addgene reference: 196971). The nucleotide sequence for SpCas9-VQR was as published (Kleinstiver B et al., Nature 523:481-485, 2015). As above, the inclusion of PuroR facilitates enrichment for successfully transfected cells following in vitro transfection, and expression of the Cas9-VQR enzyme and PuroR is driven by the CBh promoter (Gray et al., 2011 supra). Again, the plasmid also encodes the gRNA site upstream of the scaffold sequence, and gRNA expression is driven by the U6 RNA polymerase III promoter. Subsequently, custom oligonucleotide sequences corresponding to the Cas9-VQR gRNA in Table 1 (SEQ ID NO:5) were inserted into the Cas9-VQR-puro expression plasmid to generate a RHOP23H-targeting expression construct, according to standard methodologies known to those skilled in the art.

### SaCas9 Expression Construct

Initially, an expression plasmid expressing SaCas9 and PuroR was created using standard methodologies known to those skilled in the art; this construct is hereinafter referred to as SaCas9-puro. Further, SaCas9-puro was modified to alter a string of thymine (T) nucleotides in the gRNA scaffold which were predicted to negatively affect the amount of gRNA transcription; hereinafter the modified construct is referred to as SaCas9-puro-V3. In this construct, the expression of the SaCas9 enzyme and PuroR is driven by the CBh promoter (Gray et al., 2011 supra). SaCas9-puro-V3 also encodes a gRNA site upstream of the scaffold sequence, from which gRNA expression is driven by the U6 RNA polymerase III promoter. Subsequently, custom oligonucleotide sequences corresponding to the SaCas9 gRNA in Table 1 (SEQ ID NO:9) was inserted into SaCas9-puro-V3 to generate a RHOP23H-targeting expression construct. The nucleotide sequence encoding for the full-length gRNA (comprising the RhoSA1 spacer sequence and the scaffold sequence) in the SaCas9-puro V3 expression plasmid is shown below:

(SEQ ID NO: 11)
5′-GACGGGTGTGGTACGCAGCCACTgtttcagtactctggaaacagaa
tctactgaaacaaggcaaaatgccgtgtttatctcgtcaacttgttggc
gagatttttt-3′

(Note. The RhoSA1 spacer sequence is shown in uppercase, while the scaffold sequence is shown lowercase.)
Transfection of Cells with the CRISPR Expression Constructs

HEK293T cells were transfected with one each of the CRISPR expression constructs according to standard methodologies known to those skilled in the art. The cells were enriched for successfully transfected cells by culturing in the presence of puromycin. The transfected cells were grown until approximately 90% confluent, and then harvested. DNA extraction was performed according to standard methodologies known to those skilled in the art.

### Analysis of the WT Alleles

The target site, namely nucleotides −12 to 388 of exon 1 the human RHO gene, was amplified using the following primers and standard methodologies known to those skilled in the art:

Forward primer:
(SEQ ID NO: 12)
5′-GGTCAGAACCCAGAGTCATCCAGC-3′
Reverse primer:
(SEQ ID NO: 13)
5′-AGAGGTGTAGAGGGTGCTGGTGAAG-3′

The primer sequences were provided with standard overhangs (adaptor sequences) for next generation sequencing (NGS) techniques as is well-known to those skilled in the art. The indels present in WT alleles were quantified by using standard NGS (Illumina MiSeq™ System; Illumina, Inc., San Diego, CA, United States of America). The results indicated that four of the candidate gRNAs (namely, rhoNG3, rhoNG6, rhoNG8 and rhoSA1) exhibited virtually no activity at the WT allele (<3.6%), with a further two gRNAs (namely, rhoNG4 and rhoNG5) showing only a very low level of activity (6.3-7.3%) at the WT allele. In contrast, rhoNG1, rhoNG2, rhoNG7 and rho VQR showed much higher levels of activity at the WT allele ranging from 49.7% (rhoVQR) to 91.1% (rhoNG7) showing the inherent unpredictability of avoiding off-target CRISPR activities. Notably, for each of the four gRNAs showing virtually no off-target activity against the WT allele, the site of mutation within the target mutant allele corresponds to a nucleotide located at or near (e.g. within 6 or less nucleotides) of the 3′-end of the respective gRNA. It was also interesting that the results obtained with rhoNG5 and rhoVQR, which have the identical gRNA sequence, were so divergent and indicates that the SpCas9-VQR endonuclease may be less suited to applications such as this where only a disease-causing allele is to be targeted.

### Analysis of Activity at the Mutant Alleles

To assay for the specific targeting activity of the gRNAs at the RHOP23H allele sequence, CRISPR “search-and-replace” prime editing methodologies (see, for example, Anzalone A et al., Nature 576:149-157, 2019) were used to produce ADRP disease model HEK293T cells; that is, HEK293T including a RHOP23H allele. The disease model cells are denoted herein as HEK293T^{RHOP23H/+} cells. Then, in the same manner as that described above, the HEK293T^{RHOP23H/+} cells were transfected with one each of a CRISPR expression construct for each of the gRNAs, rhoNG3, rhoNG6, rhoNG8 and rhoSA1. PCR amplification of the target site and quantification of the indels present in the RHOP23H and WT alleles were quantified using the Illumina MiSeq™ system. The results are shown in FIG. 1B. Strikingly, a massive reduction in the RHOP23H alleles (from 50%) was observed for rhoNG3 (only 2.2% remaining), rhoNG6 (5.9% remaining), rhoNG8 and rhoSA1 (both with only 1.3% of the RHOP23H alleles), with edited alleles (i.e. alleles including indels) close to 50%. In contrast, WT allele frequencies remained close to 50%.

The results indicate that CRISPR-Cas systems, using Cas endonucleases which bind to an NG or NNGRRT PAM sequence and gRNAs targeted to the RHOP23H mutant allele, can be identified and developed for specific ablation of the RHOP23H mutant allele, with very little off-target activity.

### Example 2 CRISPR-Based Editing of an ADRP-Causing NR2E3 Allele

### Identification of Candidate Guide RNAs

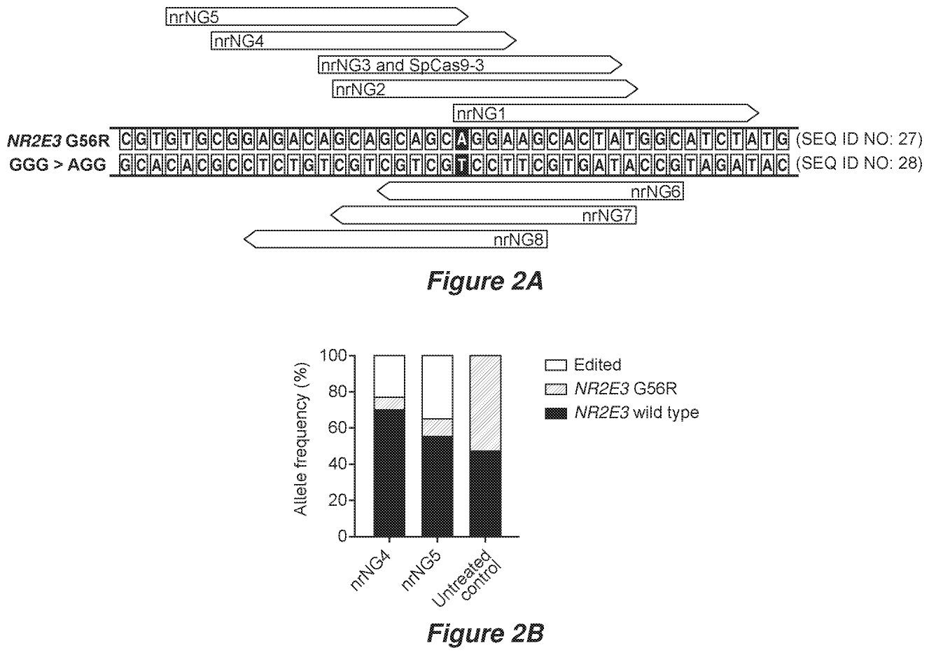

An essentially equivalent approach to that described in Example 1 was used to identify candidate gRNA sequences for allele-specific targeting of the human NR2E3G56R allele sequence. Eight (8) gRNAs for SpCas9-NG (namely, nrNG1-8) were identified, one of which is also compatible with SpCas9 (SpCas9-3) which binds to an NGG PAM. Where gRNAs of canonical length did not begin with G, an additional G was appended to the 5′ end of the gRNA to enhance transcription under the U6 RNA polymerase III promoter. In some instances, this G naturally is naturally occurring in the target sequence (SEQ ID NOs:17 and 21); otherwise, the additional G is a mismatch with the target DNA sequence (SEQ ID Nos:14, 16 and 20). These NR2E3 gRNAs are shown schematically in FIG. 2A and the allele-targeting sequences (i.e. gRNA spacer sequences) are as shown in Table 2.

TABLE 2

| gRNA | 5′→3′ targeting nucleotide sequence |
|---|---|
| nrNG1* | AGGAAGCACUAUGGCAUCUA (SEQ ID NO: 14) |
| nrNG2 | GCAGCAGCAGGAAGCACUAU (SEQ ID NO: 15) |
| nrNG3* | AGCAGCAGCAGGAAGCACUA (SEQ ID NO: 16) |
| nrNG4* | CGGAGACAGCAGCAGCAGGA (SEQ ID NO: 17) |
| nrNG5 | GUGCGGAGACAGCAGCAGCA (SEQ ID NO: 18) |
| nrNG6 | GCCAUAGUGCUUCCUGCUGC (SEQ ID NO: 19) |
| nrNG7* | AUAGUGCUUCCUGCUGCUGC (SEQ ID NO: 20) |
| nrNG8* | CUUCCUGCUGCUGCUGUCUC (SEQ ID NO: 21) |
| SpCas9-3* | AGCAGCAGCAGGAAGCACUA (SEQ ID NO: 16) |

(* An additional G nucleotide was added to the 5′ end of the given sequence to enable enhance transcription from the U6 promoter)

Screening of Candidate gRNAs for Off-Target Activity

Each of the candidate gRNA molecules were screened to ensure that their cleavage activity was limited to the NR2E3G56R allele sequence, and there was no substantial cleavage observed in the wild type (WT) allele.

### Construction of CRISPR Expression Constructs

CRISPR expression constructs were prepared in a similar manner to that described in Example 1. Briefly, custom oligonucleotide sequences encoding the gRNAs in Table 2 (SEQ ID NOs:14-21) were inserted into the Cas9-NG-puro plasmid to generate eight (8) NR2E3G56R-targeting expression constructs, according to standard methodologies known to those skilled in the art. The gRNA of SEQ ID NO:16 is also compatible with SpCas9. The plasmid for expression of SpCas9 and PuroR however, was also modified to alter a string of thymine (T) nucleotides in the gRNA scaffold which were predicted to negatively affect the amount of gRNA transcription; the modified plasmid is hereinafter referred to as SpCas9-puro-V3 and encodes a scaffold with a nucleotide sequence according to SEQ ID NO:24). Subsequently, an oligonucleotide sequence encoding the SpCas9-3 gRNA (SEQ ID NO:16) was inserted into SpCas9-puro-V3 according to standard methodologies known to those skilled in the art.

Transfection of Cells with the CRISPR Expression Constructs

HEK293T cells were transfected and enriched (for successfully transfected cells) using the same methodologies described in Example 1. The transfected cells were grown until approximately 90% confluent, and then harvested. DNA extraction was performed according to standard methodologies known to those skilled in the art.

### Analysis of the WT Alleles

The target site, namely nucleotides 18,675 to 18,985 of the NR2E3 gene (encompassing exon 2 and partial intron 1), was amplified using the following primers and standard methodologies known to those skilled in the art:

Forward primer:
(SEQ ID NO: 22)
5′-ATGCACAGTGAGGGAGACACTTC-3′
Reverse primer:
(SEQ OD NO: 23)
5′-CACCGCACTCACCTGTAGAT-3′

The primer sequences were provided with standard overhangs (adaptor sequences) for next generation sequencing (NGS) techniques as is well-known to those skilled in the art. The indels present in WT alleles were quantified by using standard NGS (Illumina MiSeq™ System). Some gRNAs were unable to edit the WT allele even when perfectly matched to the allele (i.e. gRNAs designed to target the WT allele, not NR2E3G56R); as such, they were considered inefficient and excluded from further analysis. gRNAs nrNG1, nrNG4, nrNG5 and SpCas9-3 (SEQ ID NOs:14, 17, 18 and 16) were selected for testing. The results indicated that one of the candidate gRNAs, namely nrNG4, exhibited virtually no activity at the WT allele (<1.5%), while another of the candidate gRNAs, namely nrNG5, showed only a very low level of activity (<10%) at the WT allele. In contrast, the other candidate gRNAs showed much higher levels of activity at the WT allele ranging from 36.8% (nrNG1) to 81.4% (SpCas9-3). It was interesting that the results obtained with nrNG5 and SpCas9-3 (81.4%), which have the identical gRNA sequence, were so divergent and indicates that the SpCas9 endonuclease may be less suited to applications such as this where only a disease-causing allele is to be targeted.

### Analysis of Activity at the Mutant Alleles

To assay for the specific targeting activity of the gRNAs at the NR2E3G56R allele sequence, CRISPR prime editing methodologies were used to produce ADRP disease model HEK293T cells; that is, HEK293T including a NR2E3G56R allele. The disease model cells are denoted herein as HEK293^{NR2E3G56R/+} cells. Then, in the same manner as that described above, the HEK293^{NR2E3G56R/+} cells were transfected with one each of a CRISPR expression construct for each of the gRNAs, nrNG4 and nrNG5. PCR amplification of the target site and quantification of the indels present in the NR2E3G56R and WT alleles were quantified using the Illumina MiSeq™ system. The results are shown in FIG. 2B. A massive reduction in the NR2E3G56R alleles (from 50%) was observed for nrNG4 (6.9% remaining) and nrNG5 (9.9% remaining).

The results indicate that CRISPR-Cas systems, using Cas endonucleases which bind to an NG or NGG PAM sequence and gRNAs targeted to the NR2E3G56R mutant allele, can be identified and developed for specific ablation of the NR2E3G56R mutant allele, with very little off-target activity.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be readily appreciated by those skilled in the art that the agent, pharmaceutical composition, method and uses of the present disclosure are not restricted in their use to the particular application described. Neither is the agent, pharmaceutical composition, method and uses restricted in their preferred embodiment(s) with regard to the particular elements and/or features described or depicted herein. Further, it will be readily appreciated that the agent, pharmaceutical composition, method and uses are not limited to the embodiment(s) disclosed, but are capable of numerous rearrangements, modifications and substitutions without departing from the scope of the present disclosure.

1.-26. (canceled)

27. An agent for treating a common form of autosomal dominant retinitis pigmentosa (ADRP) in a subject, wherein said agent comprises:
(i) a first nucleotide sequence encoding a CRISPR-associated (Cas) endonuclease which binds to an NG protospacer adjacent motif (PAM) sequence; and
(ii) a second nucleotide sequence encoding or comprising a guide RNA (gRNA) capable of forming a CRISPR-Cas complex with said Cas endonuclease, wherein said gRNA is specifically targeted to:
a RHOP23H target mutant allele and comprises a targeting nucleotide sequence selected from the group consisting of SEQ ID NOs:3, 6 and 8; or
a NR2E3G56R target mutant allele and comprises a targeting nucleotide sequence selected from the group consisting of SEQ ID NOs:17 and 18.

28. The agent of claim 27, wherein the first nucleotide sequence encodes an SpCas9-NG endonuclease.

29. The agent of claim 27, wherein the target mutant allele is RHOP23H and the second nucleotide sequence encodes or comprises a gRNA comprising a targeting nucleotide sequence selected from the group consisting of SEQ ID NOs:3, 6 and 8.

30. The agent of claim 29, wherein the gRNA comprises the targeting nucleotide sequence of any one of SEQ ID NOs:3 and 6 operably linked to the scaffold nucleotide sequence of SEQ ID NO:29.

31. The agent of claim 27, wherein the target mutant allele is NR2E3G56R and the second nucleotide sequence encodes or comprises a gRNA comprising a targeting nucleotide sequence selected from the group consisting of SEQ ID NOs:17 and 18.

32. The agent of claim 27, wherein the first and second nucleotide sequences are provided on one or two viral vectors.

33. The agent of claim 27, wherein the first and second nucleotide sequences are provided in a delivery vector comprising nanoparticles composed of polymeric or other organic materials.

34. A pharmaceutical composition comprising an agent according to claim 27 in combination with a pharmaceutically acceptable carrier, diluent and/or excipient.

35. The pharmaceutical composition of claim 34 suitable for administration by sub-retinal injection.

36. A method of treating a subject afflicted with, or predisposed to, autosomal dominant retinitis pigmentosa (ADRP), wherein the method comprises administering the pharmaceutical composition of claim 34 to the said subject.

37. The method of claim 36, wherein the method is commenced during adolescence.

38. The method of claim 36, wherein the method is commenced during childhood before ADRP disease onset and/or the appearance of ADRP symptoms.

39. A CRISPR-Cas complex comprising a CRISPR-associated (Cas) endonuclease which binds to an NG PAM sequence and a gRNA, wherein said gRNA is specifically targeted to:
a RHOP23H target mutant allele and comprises a targeting nucleotide sequence selected from the group consisting of SEQ ID NOs:3, 6 and 8; or
a NR2E3G56R target mutant allele and comprises a targeting nucleotide sequence selected from the group consisting of SEQ ID NOs:17 and 18.

40. The CRISPR-Cas endonuclease of claim 39, wherein the Cas endonuclease is SpCas9-NG.

41. The CRISPR-Cas complex of claim 40, wherein the gRNA comprises the targeting nucleotide sequence of any one of SEQ ID NOs:3 and 6 operably linked to the scaffold nucleotide sequence of SEQ ID NO:29.
